(19) United States
(12) Patent Application Publication
(10) Pub. No.: US 2016/0022212 A1
(43) Pub. Date: Jan. 28, 2016

(54) REUSABLE RESPIRATORY EFFORT SENSOR MODULE

(71) Applicant: DYMEDIX CORPORATION, Shoreview, MN (US)

(72) Inventors: James P. Moore, Bloomington, MN (US); Alan B. Jones, Maple Grove, MN (US); Todd M. Eiken, Lindstrom, MN (US)

(73) Assignee: DYMEDIX CORPORATION, Shoreview, MN (US)

(21) Appl. No.: 14/339,778

(22) Filed: Jul. 24, 2014

(51) Int. Cl. A61B 5/00; A61B 5/113

(52) U.S. Cl. CPC A61B 5/6831; A61B 5/1135

(57) ABSTRACT

A respiratory effort sensing belt that incorporates a PVDF transducer is constructed such that the transducer is contained within an elastic, moisture impervious plastic envelope and affixed to the envelope are a pair of cam buckles adapted to receive opposed ends of a body-encircling, single-use band. Because of the construction of the sensor module, it may be sterilized for reuse.

### CROSS REFERENCE TO RELATED APPLICATIONS

None

### BACKGROUND OF THE INVENTION

I. Field of the Invention

This invention relates generally to apparatus for monitoring respiratory activity in the course of a sleep study and more particularly to a respiratory effort sensor designed such that the sensor electronics and associated leads for connecting the electronics to a polysomnograph machine (PSG) is adapted for connection to a body-encircling band and remains reusable and only the low-cost, body-encircling band position is disposable following a use thereof.

II. Discussion of the Prior Art

In published U.S. Application 2008/0275356, there is described a respiratory effort belt that has been marketed by Dymedix Corporation of Shoreview, Minn. for a number of years. It comprises a PVDF film member having first and second metalized major surfaces to which a pair of approximately six foot long tabbed leads is electrically connected. The resulting assembly is permanently affixed to the surface of a body-encircling belt member. Signals are generated when the PVDF film on the belt member is stressed due to stretching as the user breathes in and out. The leads connect to a PSG, allowing the signals to be used in diagnosis and analysis.

Approximately 95% of the manufacturing cost of the respiratory effort belt resides in its sensor electronics and associated leads while the remaining 5% of the cost resides in the body-encircling belt on which the sensor electronics are affixed.

For hygienic reasons, most hospital sleep labs dispose of the respiratory effort belts after a use on a given patient. It is, of course, advantageous to reduce the cost of the diagnostic procedure and the present invention achieves this desired result.

### SUMMARY OF THE INVENTION

In accordance with the present invention, there is provided a respiratory effort belt comprising a PVDF film strip having a layer of metallization on opposed major surfaces thereof. First and second insulated conductive leads, each with first and second ends, have the first end of the first lead electrically connected to the layer of metallization on one of the opposed major surfaces and the second lead electrically connected to the layer of metallization on another of the opposed major surfaces. A flexible, elastic, moisture impervious, generally rectangular envelope is made to contain the metalized PVDF film strip and only a distal end portion of the first and second conductive leads. A pair of connectors is releasably attached individually to the first and second ends of the envelope. A disposable elastic or inelastic band, whose length is sufficient to encircle a human's torso, is adapted to be selectively joined and released to and from the pair of connectors. This allows the band to be disposed of after use while the envelope, with its connectors and containing the sensor element and leads, remains reusable following sterilization.

### DESCRIPTION OF THE DRAWINGS

The foregoing features, objects and advantages of the invention will become apparent to those skilled in the art from the following detailed description of a preferred embodiment, especially when considered in conjunction with the accompanying drawings in which like numerals in the several views refer to corresponding parts.

FIG. 1 is a partial frontal view of a respiratory effort belt comprising a preferred embodiment of the present invention; and

FIG. 2 is an exploded view of the reusable sensor module.

### DESCRIPTION OF THE PREFERRED EMBODIMENTS

This description of the preferred embodiments is intended to be read in connection with the accompanying drawings, which are to be considered part of the entire written description of this invention. In the description, relative terms such as “lower”, “upper”, “horizontal”, “vertical”, “above”, “below”, “up”, “down”, “top” and “bottom” as well as derivatives thereof (e.g., “horizontally”, “downwardly”, “upwardly”, etc.) should be construed to refer to the orientation as then described or as shown in the drawings under discussion. These relative terms are for convenience of description and do not require that the apparatus be constructed or operated in a particular orientation. Terms such as “connected”, “connecting”, “attached”, “attaching”, “join” and “joining” are used interchangeably and refer to one structure or surface being secured to another structure or surface or integrally fabricated in one piece, unless expressively described otherwise.

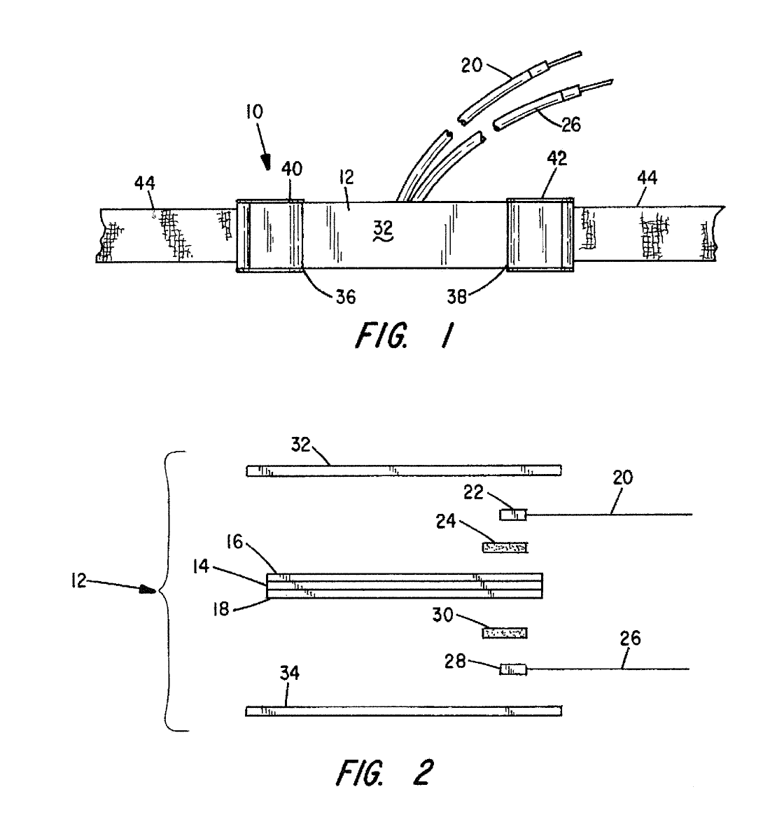

Referring first to FIG. 1, there is indicated generally by numeral 10 a respiratory effort belt constructed in accordance with the present invention. It is seen to comprise a reusable, sterilizable sensor module 12, the construction of which is best illustrated in the exploded view of FIG. 2. In FIG. 2, a strip of polyvinylidine fluoride (PVDF) is identified by numeral 14. The strip is generally rectangular and deposited on the opposed major surfaces thereof are metallization layers 16 and 18. An electrical lead 20 has a conductive terminal pad 22 bonded to the metallization layer 16 by a conductive adhesive member 24. Likewise, an electrical lead 26 has its conductive terminal pad 28 conductively affixed to the metallization layer 18 by means of a conductive adhesive layer 30.

The PVDF film 14, as well as distal end portions of the leads 20 and 26, is contained within a moisture impervious plastic envelope comprising elastic polymer layers 32 and 34 that are laminated together about the perimeters thereof to form the envelope.

Without limitation, the resulting module 12 may be about 5 inches in length and about 1½ inches in width.

Referring again to FIG. 1, affixed to the end portions 36 and 38 of the envelope comprising the module 12 are connectors 40 and 42 that are used to releasably couple the module 12 to a body-encircling band 44. Without limitation, the connectors 40 and 42 preferably comprise cam buckles, many types of which are commercially available. Such cam buckles releasably clamp to the opposed ends 36 and 38 of the envelope comprising the module 12 and permit adjustment of the body-encircling band 44 to a desired degree of snugness.

It is contemplated that the body-encircling band 44 be of an inexpensive cloth fabric that is relatively inelastic.

In use, the respiratory effort belt 10 will be placed about a patient's torso, either about the chest or abdomen, and any slack taken up by tensioning the band with the cam buckle 40 or 42 open and when the tension is deemed appropriate, the cam lever is released, locking the band 44 from moving relative to the module 12. As the subject breathes, the envelope comprising the sensor module 12, being elastic, will stretch upon inspiration and shrink during expiration. This places corresponding stresses on the PVDF film element 14 thereby producing, via the piezoelectric effect, electrical output signals on the leads 20 and 26.

Upon completion of a patient's sleep study, the body-encircling band 44 can be separated from the buckles 40 and 42 and properly disposed of. The module 12, however, can be retained for reuse following sterilization and/or cleaning and only the relatively inexpensive band 44 needs replacement. In that the great majority of the cost resides in the module 12 and because this module is reusable, a substantial savings results for the consumer who need only replace the fabric belt 44 and not the entire respiratory effort belt each time the belt is used.

This invention has been described herein in considerable detail in order to comply with the patent statutes and to provide those skilled in the art with the information needed to apply the novel principles and to construct and use such specialized components as are required. However, it is to be understood that the invention can be carried out by specifically different equipment and devices. Also, various modifications, both as to the equipment and operating procedures, can be accomplished without departing from the scope of the invention itself.

What is claimed is:

1. A respiratory effort belt comprising:
a) a PVDF film strip having a layer of metallization on opposed major surfaces thereof;
b) first and second insulated leads each with first and second ends, the first end of the first lead electrically connected to the layer of metallization on one of the opposed major surfaces and the second lead electrically connected to the layer of metallization on another of the opposed major surfaces;
c) a flexible, elastic, moisture impervious, generally rectangular elastomeric envelope containing the PVDF film strip and only a distal end portion of the first and second conductive leads, the envelope having first and second end portions; and
d) a pair of connectors releasably attached individually to said first and second end portions of the envelope and adapted to be releasably affixed to opposed ends of a disposable band of a length sufficient to encircle a human's torso.

2. The respiratory effort belt of claim 1 wherein the connectors comprise cam buckles.

3. The respiratory effort belt of claim 1 wherein the band comprises a web fabric.
